(19) United States
(12) Patent Application Publication
(10) Pub. No.: US 2024/0210343 A1
(43) Pub. Date: Jun. 27, 2024

(54) NANOPORE SENSING DEVICE WITH MULTIPLE SENSING LAYERS

(71) Applicant: IMEC VZW, Leuven (BE)

(72) Inventors: Anne Verhulst, Houtvenne (BE); Pol Van Dorpe, Spalbeek (BE); Liesbet Lagae, Leuven (BE); Cedric Huyghebaert, Heverlee (BE)

(21) Appl. No.: 18/392,429

(22) Filed: Dec. 21, 2023

(30) Foreign Application Priority Data
Dec. 23, 2022 (EP) 22216619.1

(51) Int. Cl. G01N 27/12

(52) U.S. Cl. CPC G01N 27/128

(57) ABSTRACT

In a first aspect, a nanopore sensing device is provided that includes: (i) a nanopore having a first orifice and second orifice, and a length running from the first to the second orifice; and (ii) one or more sensors for sensing an electric feature in the nanopore; wherein the nanopore sensing device comprises a plurality of sensing layers arranged along the length, each sensing layer being part of one of the sensors and each adjacent pair of sensing layers being separated by an isolating layer, and at least one of the sensors is a field-effect transistor.

### CROSS-REFERENCE TO RELATED APPLICATIONS

The present application is a non-provisional patent application claiming priority to European Patent Application No. EP 22216619.1, filed on Dec. 23, 2022, the contents of which are hereby incorporated by reference.

### FIELD OF THE DISCLOSURE

The present disclosure relates to nanopore sensing devices and in some examples to nanopore sensing devices based on a field-effect transistor.

### BACKGROUND

Biosensors developed during the last decades have enabled a wide set of applications in life sciences. Among the different types of biosensors, nanopore sensors offer single particle sensitivity, thereby facilitating characterization of individual (bio)molecules—which was before essentially restricted to fluorescence spectroscopy and force spectroscopy. Accordingly, they have received considerable attention and a substantial number of device architectures therefor have been proposed and developed. Today, nanopore sensors are at the heart of state-of-the-art systems for DNA sequencing, and researchers are now setting their sights on proteomics, which is an even more challenging task than DNA sequencing.

While biological nanopores still outperform their solid-state counterparts for DNA-based applications, the latter offer a variety of benefits for more complex single-particle sensing. This includes the ability to tailor the size, shape, and surface properties of the solid-state pore for improved sensing; a formation process that is not stochastic and that results in devices that are physically and chemically more robust than their biological counterparts; and more straightforward co-integration with electronic devices.

### SUMMARY

Nanopore field-effect transistors (nanopore FETs), in which the nanopore runs through or near the channel of the FET, can be configured such that the electrical conditions in the nanopore modulate the conductivity of the channel. As such, when a particle passes through the nanopore, it can influence the source-drain current through the channel. Thus, a nanopore FET sensor can be formed wherein translocation of a particle through the nanopore affects the gating of the FET, which can in turn be used to detect and (at least partially) characterize the particle. Such a nanopore FET can have a unique boosting effect, thereby increasing the sensitivity up to 1000% and above.

However, sensing non-DNA particles is challenging due, in part, to the speed of the particles. While a plethora of molecular machines (e.g., enzymes) are available to reduce and control the speed with which DNA molecules pass through a nanopore, such velocity-reduction mechanisms are not readily available for other particles, e.g., proteins. Though nanopore FETs have an edge with regard to detecting fast translocating particles—in part thanks to a larger detection bandwidth owing to the significantly larger electronic current used for sensing (as opposed to e.g. the ionic current used in a biological-nanopore detection system)—, there is another aspect to the speed issue. In addition to the challenges posed by high (average) particle speed, that speed is also not controlled and is unknown. As such, a signal measured from a nanopore sensor may come from, e.g., a longer fast-moving particle or a shorter slow-moving one, with significant difficulty in distinguishing between the possibilities.

The present disclosure provides improved nanopore sensing devices that can facilitate the determination of the speed and/or length of an analyte longitudinal to the nanopore. The present disclosure also provides systems and methods associated therewith.

In some embodiments of the present disclosure an analyte can be detected and/or (at least partially) characterized with good sensitivity. In some embodiments of the present disclosure that an electric fingerprint of the analyte can be determined.

In some embodiments of the present disclosure the FET sensor can be operated such as to benefit from a considerable ‘boosting effect’ in its sensitivity. In some embodiments of the present disclosure the FET sensor can have a relatively high signal to noise ratio.

In some embodiments of the present disclosure the speed of an analyte longitudinal to the nanopore can be determined. In some embodiments of the present disclosure the length of an analyte longitudinal to the nanopore can be determined. In some embodiments of the present disclosure these can be determined with relatively high accuracy.

In some embodiments of the present disclosure the analyte can translocate through the nanopore at a substantially constant speed.

In some embodiments of the present disclosure the nanopore sensing device can have a relatively high output signal bandwidth. In some embodiments of the present disclosure the high output signal bandwidth can be supported by a correspondingly large-bandwidth readout circuit.

In some embodiments of the present disclosure measures can be taken to reduce the complexity of manufacturing the nanopore sensing device. For example, in some embodiments of the present disclosure a FET sensor can be combined with a sensing layer and/or sensor which benefit the manufacturability of the sensing device as a whole. In some embodiments of the present disclosure two or more sensing layers can share one or more contacts in common.

In some embodiments of the present disclosure the sensing layers may be closely about the nanopore, thereby maximizing their sensitivity to the nanopore's contents.

Some embodiments of the present disclosure provide a relatively high throughput of analyte analysis. In some embodiments of the present disclosure this throughput can be increased by configuring multiple nanopore sensing devices in parallel. In some embodiments of the present disclosure the nanopore sensing device can have a relatively small footprint, further increasing its ability to be parallelized.

In some embodiments of the present disclosure the nanopore sensing device can be employed for sensing a variety of analytes, including polypeptides and/or proteins.

The lack of speed and/or length determination of an analyte in prior sensors diminishes the potential of nanopore FETs. To meet this need, the nanopore sensing device described herein includes multiple sensing layers and at least one FET sensor, thereby allowing the speed and/or length of the analyte longitudinal to the nanopore to be detected by detecting the analyte at the different sensing layers as it is translocating through the nanopore, while maintaining a highly sensitive reading of the analyte using the FET sensor.

In a first aspect, the present disclosure relates to a nanopore sensing device, comprising: (i) a nanopore having a first orifice and second orifice, and a length running from the first to the second orifice; and (ii) one or more sensors for sensing an electric feature in the nanopore; wherein the nanopore sensing device comprises a plurality of sensing layers arranged along the length, each sensing layer being part of one of the sensors and each adjacent pair of sensing layers being separated by an isolating layer, and at least one of the sensors is a field-effect transistor.

In a second aspect, the present disclosure relates to a system comprising the nanopore sensing device according to any embodiment of the first aspect and a microfluidic system coupled to the nanopore sensing device.

In a third aspect, the present disclosure relates to a method for sensing an analyte, comprising: (a) providing an analyte in a nanopore sensing device as defined in any embodiment of the first or second aspect; (b) translocating the analyte through the nanopore; and (c) sensing an electric feature in the nanopore as the analyte translocates therethrough.

In a fourth aspect, the present disclosure relates to a use of a nanopore sensing device as defined in any embodiment of the first aspect, for determining a speed and/or length of an analyte longitudinal to the nanopore.

Aspects of the present disclosure are set out in the accompanying independent and dependent claims. Features from the dependent claims may be combined with features of the independent claims and with features of other dependent claims as appropriate and not merely as explicitly set out in the claims.

Although there has been constant improvement, change and evolution of devices in this field, the present concepts are believed to represent substantial new and novel improvements, including departures from prior practices, resulting in the provision of more efficient, stable and reliable devices of this nature.

The above and other characteristics, features and benefits of the present disclosure will become apparent from the following detailed description, taken in conjunction with the accompanying drawings, which illustrate, by way of example, the principles of the present disclosure. This description is given for the sake of example only, without limiting the scope of the present disclosure. The reference figures quoted below refer to the attached drawings.

### BRIEF DESCRIPTION OF THE FIGURES

The above, as well as additional features and benefits, may be understood through the following illustrative and non-limiting detailed description, with reference to the appended drawings. In the drawings like reference numerals will be used for like elements unless stated otherwise.

The above described aspects and implementations are further explained in the following description of embodiments with respect to the following drawings:

FIG. 1 schematically depicts a nanopore sensing device, comprising two sensing layers: one being part of a FET and the other being a conductor layer, in accordance with an example embodiment.

FIG. 2 schematically depicts a nanopore sensing device comprising three sensing layers: two being part of a FET and the third being a conductor layer, in accordance with an example embodiment.

FIG. 3 schematically depicts the signals one might obtain as an analyte translocates through a nanopore sensing device as in FIG. 2, in accordance with an example embodiment.

FIG. 4 schematically depicts a nanopore sensing device comprising two sensing layers: one being part of an extended-gate FET and the other being a conductor layer, in accordance with an example embodiment.

FIG. 5 schematically depicts a nanopore sensing device comprising two sensing layers: both being part of a FET and sharing a common drain, in accordance with an example embodiment.

FIG. 6 schematically depicts a nanopore sensing device comprising three sensing layers: two being part of a FET and sharing a common source and drain, the third being a conductor layer, in accordance with an example embodiment.

In the different figures, the same reference signs refer to the same or analogous elements.

All the figures are schematic, not necessarily to scale, and generally only show parts to elucidate example embodiments, wherein other parts may be omitted or merely suggested.

### DETAILED DESCRIPTION

The present disclosure will be described with respect to particular embodiments and with reference to certain drawings but the scope of the present disclosure is not limited thereto. The drawings described are only schematic and are non-limiting. In the drawings, the size of some of the elements may be exaggerated and not drawn to scale for illustrative purposes. The dimensions and the relative dimensions do not correspond to actual reductions to practice of the present disclosure.

Furthermore, the terms first, second, third and the like in the description and in the claims, are used for distinguishing between similar elements and not necessarily for describing a sequence, either temporally, spatially, in ranking or in any other manner. It is to be understood that the terms so used are interchangeable under appropriate circumstances and that the embodiments of the present disclosure described herein are capable of operation in other sequences than described or illustrated herein.

Moreover, the terms top, bottom, over, under and the like in the description and the claims are used for descriptive purposes and not necessarily for describing relative positions. It is to be understood that the terms so used are interchangeable with their antonyms under appropriate circumstances and that the embodiments of the present disclosure described herein are capable of operation in other orientations than described or illustrated herein.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. The term “comprising” therefore covers the situation where only the stated features are present and the situation where these features and one or more other features are present. Thus, the scope of the expression “a device comprising means A and B” should not be interpreted as being limited to devices consisting only of components A and B. It means that with respect to the present disclosure, the only relevant components of the device are A and B.

Similarly, it is to be noticed that the term “coupled”, also used in the claims, should not be interpreted as being restricted to direct connections only. The terms “coupled” and “connected”, along with their derivatives, may be used. It should be understood that these terms are not intended as synonyms for each other. Thus, the scope of the expression “a device A coupled to a device B” should not be limited to devices or systems wherein an output of device A is directly connected to an input of device B. It means that there exists a path between an output of A and an input of B which may be a path including other devices or means. “Coupled” may mean that two or more elements are either in direct physical or electrical contact, or that two or more elements are not in direct contact with each other but yet still co-operate or interact with each other.

Reference throughout this specification to “one embodiment” or “an embodiment” means that a particular feature, structure or characteristic described in connection with the embodiment is included in at least one embodiment of the present disclosure. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly, it should be appreciated that in the description of exemplary embodiments of the present disclosure, various features of the present disclosure are sometimes grouped together in a single embodiment, figure, or description thereof for the purpose of streamlining the disclosure and aiding in the understanding of one or more of the various aspects thereof. This method of disclosure, however, is not to be interpreted as reflecting an intention that the claimed invention requires more features than are expressly recited in each claim. Rather, as the following claims reflect, claimed subject matter lies in less than all features of a single foregoing disclosed embodiment. Thus, the claims following the detailed description are hereby expressly incorporated into this detailed description, with each claim standing on its own as a separate embodiment.

Furthermore, while some embodiments described herein include some but not other features included in other embodiments, combinations of features of different embodiments are meant to be within the scope of the present disclosure, and form different embodiments, as would be understood by those in the art. For example, in the following claims, any of the claimed embodiments can be used in any combination.

In the description provided herein, numerous specific details are set forth. However, it is understood that embodiments of the present disclosure may be practised without these specific details. In other instances, well-known methods, structures and techniques have not been shown in detail in order not to obscure an understanding of this description.

Reference will be made to transistors. These are devices having a first main terminal (e.g. a source or collector), a second main terminal (e.g. a drain or emitter) and a control terminal (e.g. a gate or base) for controlling the flow of electrical charges between the first and second main terminals.

The following terms are provided solely to aid in the understanding of the present disclosure.

As used herein, and unless otherwise specified, a field-effect transistor (FET) is a type of transistor wherein an electric field controls the current flow between the source and drain through a semiconductor. A voltage applied at the third—e.g. “gate”—terminal can create and affect the size and shape of a conductive channel in the semiconductor, thereby controlling the flow of charge carriers between the source and drain. Structurally, a FET thus comprises a semiconductor as active (channel) layer. Functionally, a FET can have a subthreshold swing at room temperature (e.g. 20° C.) of 150 mV/dec (millivolt per decade) or less. Note that while a FET can include different regions and terminals, there need not be a strict junction between (all of) these. Indeed, there exist so-called ‘junction-less FETs’ wherein a source, channel and drain region are present in a single body (e.g. a single nanowire). Note that while a FET often comprises a source and drain region with identical doping type (e.g. both p-type or both n-type), this is not required in all embodiments. Indeed, there e.g. exist so-called ‘tunnel FETs’ wherein the source and drain region have opposite doping type, and whereby the gate not only affects the size and shape of the conductive channel, but also locally (i.e. at the source-channel junction) sets the tunnel barrier height and width. Accordingly, it may be the source-channel junction which is dominant in determining the current in the subthreshold region. A tunnel FET can achieve subthreshold swings at room temperature far below 60 mV/dec, essentially without lower limit. Note moreover that while a FET can include one semiconductor as channel material, there need not be a limitation to one material. Indeed, there exist so-called ‘heterostructure stacks’ wherein the channel longitudinally (i.e. from source to drain) or laterally (i.e. parallel paths between source and drain) comprises stacked layers of semiconductor material. Such selections may be targeted at adding strain in the FET which affects the mobility, or at creating more favourable conduction through the channel by lowering the inherent tunnel barrier (e.g. for tunnel FETs).

As used herein, and unless otherwise specified, a nanopore field-effect transistor is a type of FET in which the role of the gate is effectively fulfilled by the nanopore. Accordingly, it is the electric potential profile in the nanopore which modulates the current between the source and drain. In turn the electric potential profile in the nanopore is affected by the content of the nanopore—including e.g. an analyte translocating therethrough—, and more specifically by the content's electric features (e.g. electric charge and/or electric dipole, making up the (local) electric field/electric potential variations inside or near the chemical entities in the content). As such, (changes in) the source-drain current can be used to detect and (at least partially) characterize an electric feature of the analyte, and by extension the analyte as a whole.

As used herein, and unless otherwise specified, an extended-gate field-effect transistor is a type of FET which has a ‘main body’ (e.g. with a source, drain, channel and gate) like a traditional FET, but wherein the gate is electrically (e.g. resistively) coupled to a (highly) conductor layer (e.g. a 2D conductor layer—such as graphene—or a metal—such as Pt, Ru, W, Al, or Cu). The combination of a gate and conductor layer may then also be referred to as a ‘long gate’. In some embodiments, the gate may be electrically extended by means of the conductor layer as such. Alternatively, the gate may be electrically connected to the conductor layer by means of a further conductive material.

As used herein, and unless otherwise specified, sensing layers which have all their contacts in common yield a single output signal and are thus considered to be part of a single sensor. Conversely, sensing layers having at least one dedicated contact (e.g. one shared and one dedicated or both dedicated) can yield two output signals and are thus considered to be part of two separate sensors.

In a first aspect, the present disclosure relates to a nanopore sensing device, comprising: (i) a nanopore having a first orifice and second orifice, and a length running from the first to the second orifice; and (ii) one or more sensors for sensing an electric feature in the nanopore; wherein the nanopore sensing device comprises a plurality of sensing layers arranged along the length, each sensing layer being part of one of the sensors and each adjacent pair of sensing layers being separated by an isolating layer, and at least one of the sensors is a field-effect transistor.

In some embodiments, the field-effect transistor may have a subthreshold swing at room temperature of 150 mV/dec or less; for example 120 mV/dec or less, v100 mV/dec or less, for example 80 mV/dec or less, for example 70 mV/dec or less, for example 60 mV/dec or less. In some embodiments, the nanopore sensing device may be operated such (e.g. the cis and/or trans electrode may be biased such) that the field-effect transistor is operated in its subthreshold regime. Operating the field-effect transistor in its subthreshold regime allows it to have an exceedingly high sensitivity (e.g. due to a ‘boosting effect’). This is can be beneficial, for example when the goal is not only to detect but also to characterize the analyte (cf. infra).

In some embodiments, the field-effect transistor may be a nanopore field-effect transistor or an extended-gate field-effect transistor. In some embodiments, the field-effect transistor may comprise a sensing layer which is a conductive or semiconductive part of the field-effect transistor. For a nanopore FET, the sensing layer may for example be the (semiconductive) channel of the FET. For an extended-gate FET, the sensing layer may for example be the conductor layer coupled to the gate (i.e. as part of the extended/long gate; and thus capacitively coupled to the channel). As such, the gating effect of the nanopore on the channel may be direct or indirect.

In the case of an extended-gate FET, the main body may be located (very) close to (e.g. neighbouring)—but separated from (e.g. the latter does not go through it)—the nanopore. One benefit of an extended-gate FET may be that it can be easier to manufacture a FET with a gate extending to a conductor layer as sensing layer, compared to e.g. a nanopore field-effect transistor wherein the channel itself is the sensing layer.

Regardless of the nature of the field-effect transistor (e.g. nanopore FET or extended-gate FET), a difference in signal from the FET may be substantially (e.g. exclusively) directly or indirectly (cf. supra) due to the gating effect of the nanopore (i.e. due to a change within the nanopore, such as the translocation of an analyte therethrough). In some embodiments using an extended-gate FET, the gate may be biased (i.e. not by the contents of the nanopore; but e.g. by an external voltage), so that a measured signal may be at least partially due to the bias (but a difference in signal remains substantially—for example exclusively—due to the gating effect of the nanopore). Notwithstanding, in some embodiments (using a FET of any nature), the gate may not be biased. Accordingly, any measured signal may in some embodiments be substantially—for example exclusively—due to the gating effect of nanopore.

In some embodiments, the nanopore may run through—or at least near (sufficiently to have—directly or indirectly—a gating effect on the channel)—the sensing layer. In some embodiments, the nanopore may be at least partially surrounded by the sensing layers. In some embodiments, the nanopore may be fully (i.e. 360°) surrounded by the sensing layer. In other words: the sensing layer may be at least partially—for example fully—about the nanopore. The closer the sensing layer surrounds the nanopore, the greater the gating effect of the nanopore—and therefore the greater the signal and signal-to-noise ratio—can be. In some embodiments, the sensing layers and the isolating layers may form a stack and the nanopore may (completely) cross through the stack.

The sensing layers may be substantially parallel to one another. In some embodiments, the nanopore may be oriented at an angle (different from 0; e.g. the length may be oriented at the angle) to the sensing layers. The angle may for example be from 30 to 150°, for example from 60 to 120°, for example from 85 to 95°, for example 90°. Thus, if the sensing layers are considered to be oriented horizontally (i.e. they are substantially parallel to the horizontal plane), the nanopore may be substantially vertical (i.e. the length may extend vertically). Accordingly, the sensing layers (and isolating layer(s)) may be stacked substantially vertically. In some embodiments, the source and a region may define a source-drain axis and the nanopore may be oriented at an angle (different from 0; e.g. the length may be oriented at the angle) to the source-drain axis. The angle may again for example be from 30 to 150°, for example from 60 to 120°, for example from 85 to 95°, for example 90°. Thus, the source-drain axis—which can be parallel to the sensing layers (for example to the one sensing layer forming the FET sensor with the source and drain)—may be perpendicular to the nanopore. If the nanopore is considered to be vertical, such a FET may then be referred to as a ‘horizontal FET’ (e.g. as opposed to a ‘vertical FET’, where the source-drain axis is parallel to nanopore). Horizontal FETs (and horizontal sensing layers/sensors in general) can be beneficial in that this orientation can take up the least amount of space along the nanopore length, thereby allowing to efficiently stack the plurality of sensing layers (and sensors) without using an excessively long nanopore.

In general, the sensing layers may be semiconductor layers and/or conductor layers. For example, each sensing layer may independently be a semiconductor layer or a conductor layer.

At least the sensing layer comprised in the field-effect transistor can be a semiconductor layer. In some embodiments, one or more further sensing layers may be semiconductor layers. In some embodiments, each semiconductor layer may comprise a semiconductor material independently selected from group IV (e.g. Si, Ge, or SiGe), III-V (e.g. GaAs, InGaAs, or InP), 2D (e.g. a transition metal dichalcogenide, graphene or hexagonal boron nitride) semiconductors or semiconducting carbon nanotubes (CNTs). In some embodiments, the semiconductor layer may comprise more than one semiconductor material (e.g. a stack of semiconductor materials, like a heterostructure semiconductor stack). As such, the semiconductor channel material may be different from the source material and/or from the drain material (e.g. forming a heterojunction at the source-channel and/or channel-drain junction).

In some embodiments, at least one of the sensing layers may be a conductor layer. In some embodiments, each conductor layer may comprise a conducting material independently selected from 2D conductors (e.g. graphene), metals (e.g. Pt, Ru, W, Al, or Cu) or metallic CNTs. In some embodiments, the conductor layer may be part of a conductive sensor (e.g. comprising the conductive sensing layer and two contacts to the sensing layer) or an extended-gate field-effect transistor (cf. supra). Sensors comprising a conductor layer as sensing layer can have in common that they are easier to fabricate than those based on semiconductor layers and may in some instances thus be chosen for that reason. Similar to a FET, in a nanopore conductive sensor, the resistance/conductance through the active layer (i.e. here the conductive sensing layer) is modulated by the contents of the nanopore. However, this effect can be much (e.g. several orders of magnitude) smaller in a conductor layer than it can be in a FET, so that conductive sensors can be considerably less sensitive than FET sensors. Hence, it is contemplated by the present disclosure that at least one of the sensors is a FET, so that at least one highly sensitive sensor is present (and thus the signal thereof can e.g. be used to characterize the analyte). Notwithstanding, while sometimes insufficient for e.g. characterization of the analyte, conductive sensors may be adequate to detect the presence of the analyte and thus allow to use this information (e.g. in combination with that of the FET sensor) to determine for instance the speed and/or length of the analyte.

In some embodiments, the isolating layer may be an insulating (i.e. non-conducting) layer. In some embodiments, the isolating layer may comprise a dielectric, such as SiO₂.

In some embodiments, the isolating layers may have a thickness of between 2 nm and 100 nm; for example between 3 nm and 50 nm, for example between 4 nm and 25 nm, for example between 5 nm and 10 nm.

Depending on the nature of the sensing layer (e.g. in view of their stability when in contact with the nanopore's contents), there may be a need and/or desire to further provide a thin protective layer between the sensing layer and the (inside of) the nanopore. This is for example sometimes the case for Si, while e.g. Pt is in many examples stable regardless. In some embodiments, the protective layer may for example be an oxide (e.g. SiO₂ or RuO₂) or nitride (e.g. SiN) of (one of) the sensing layers. The protective layer can be sufficiently thin so as to not compromise the sensitivity of the sensing layer to the nanopore's contents too much; for example, the protective layer may have an ‘effective oxide thickness’ (i.e. relative to SiO₂) of 3 nm or below, for example 2 nm or below, for example 1 nm or below. Such a protective layer may also be beneficial to make the nanopore more uniform (e.g. with respect to nature/surface charge, but also in width); cf. infra. Accordingly, not only one or more sensing layers but a substantial part (e.g. at least 50%, for example at least 70%, for example at least 90%) of the section between the uppermost sensing layer and the lowermost sensing layer section may be lined with the protective layer.

In the case of the extended gate, that oxide (e.g. for Ru) could be Ru-oxide, and some materials do not need an oxide (Pt) but will be stable in a metal-fluid connection.

In some embodiments, the nanopore may have a length of from 1 nm to 1 μm, for example from 2 nm to 200 nm, for example from 5 nm to 100 nm, for example from 10 nm to 50 nm.

In some embodiments, the nanopore may have a width of between 1 and 200 nm; for example between 3 and 100 nm, for example between 5 nm and 30 nm. The width may be the widest dimension of a cross-section through the nanopore. In some embodiments, the width may be the maximal width or the average width. In some embodiments wherein the nanopore has a circular cross-section (i.e. the nanopore is cylindrical), the width may be the diameter of the nanopore. That said, the nanopore is not limited by its cross-section, and the cross-section may for example be circular, oval, triangular, rectangular, square, pentagonal, hexagonal, etc. Notwithstanding, a circular cross-section may in some instances nevertheless be used, as it may allow for the highest sensitivity for the sensing layers (for example if the analyte is in first approximation rather spherical or cylindrical).

The nanopore may have a section defined between an uppermost sensing layer (e.g. a top thereof) and a lowermost sensing layer (e.g. a bottom thereof). In some embodiments, the section may have a substantially constant width. In some embodiments, the section may have substantially uniform sidewalls. In some embodiments, the section may have both a substantially constant width and substantially uniform sidewalls. A nanopore that is substantially uniform (e.g., in width and nature) across the sensing region (i.e. across the section between the uppermost and lowermost sensing layers) can be beneficial in that it can promote the analyte having a constant speed throughout the sensing region. In some embodiments, the width may be substantially constant if any local width differs from the average width by less than 20%, for example less than 15%, for example less than 10%. In some embodiments, the sidewalls may be substantially uniform may be substantially uniform if the sidewalls have a substantially uniform surface charge. In some embodiments, the surface charge may be substantially uniform if any local surface charge differs from the average surface charge by less than 15%, for example less than 10%, for example less than 5%. For example, the sidewalls may be made up of substantially the same material.

In alternative embodiments, the nanopore may have a smaller width at the sensing layers. Such a nanopore may be well suited the sensing longer analytes, as it reduces the total resistance in the nanopore, which has a positive impact on the overall signal-to-noise ratio of the nanopore sensing device.

In some embodiments, the nanopore may have an above average (i.e. wider) width above the uppermost sensing layer and/or below the lowermost sensing layer. This can aid in facilitating entry into and evacuation out of the nanopore for the analyte.

In some embodiments, at least two of the sensing layers may have one or two contacts in common. In some embodiments, the one or two common contacts may be a common source and/or a common drain. Sharing contacts may improve the ease with which the sensors can be fabricated. Note that in the case where sensing layers share all their contacts, they will also output a single signal. Nevertheless, while it adds some complication, it is possible (e.g. during post-processing of the signal) to deconvolve the signal into signals for the individual sensing layers, or at least to derive the relevant sensing events at the different sensing layers from the convoluted signal. This situation could therefore be regarded as a kind of trade-off between ease of manufacture and ease of signal processing.

In some embodiments, the nanopore sensing device may further comprise a readout circuitry. In some embodiments, the readout circuitry may comprise an amplifier. A readout circuitry may for example comprise one or more further field-effect transistors. Notwithstanding, it will be understood that such FETs are distinct from—and not to be confounded with—the at least one sensor which is a FET as defined in the first aspect. Indeed, such further FETs do not comprise a sensing layer (sensitive to an electric feature in the nanopore) arranged along the length of the nanopore.

In some embodiments, the readout circuit may have a bandwidth of at least 10 kHz; for example at least 100 kHz; for example at least 500 kHz, for example at least 1 MHz. Nanopore sensing devices in accordance with the present disclosure can have a relatively high readout bandwidth. In some embodiments, the sensors may output one or more signals at the aforementioned bandwidth (combined or individually).

In some embodiments, the field-effect transistor may have a signal-to-noise ratio of at least 0 dB up to at least 100 kHz; for example at least 1 MHz, for example at least 10 MHz. The field-effect transistor in nanopore sensing device in accordance with the present disclosure can have a relatively high sensitivity (and thus signal-to-noise ratio). This may for example be at least in part thanks to the ‘boosting’ effect achieved when operating the field-effect transistor in its subthreshold regime.

In some embodiments, any feature of any embodiment of the first aspect may independently be as correspondingly described for any embodiment of any of the other aspects.

In a second aspect, the present disclosure relates to a system comprising the nanopore sensing device according to any embodiment of the first aspect and a microfluidic system coupled to the nanopore sensing device.

The microfluidic system may for example comprise a plurality of microfluidic channels. In some embodiments, the microfluidic system may further comprise a (microfluidic) pump for generating and/or controlling a fluid flow through the microfluidic system.

In some embodiments, the system may further comprise: (iii) a cis reservoir and a trans reservoir; and (iv) a cis electrode and a trans electrode, for generating a potential difference for translocating an analyte through the nanopore.

In some embodiments, the system may further comprise a control unit. The control unit may for example be configured for controlling the nanopore sensing device and/or the microfluidic system. In some embodiments, the control unit may be configured for performing step c of the method according to the third aspect (cf. infra).

In some embodiments, any feature of any embodiment of the second aspect may independently be as correspondingly described for any embodiment of any of the other aspects.

In a third aspect, the present disclosure relates to a method for sensing an analyte, comprising: (a) providing an analyte in a nanopore sensing device as defined in any embodiment of the first or second aspect; (b) translocating the analyte through the nanopore; and (c) sensing an electric feature in the nanopore as the analyte translocates therethrough.

In some embodiments, providing the analyte in the nanopore sensing device may comprise providing a dispersion (e.g. a solution, colloid or suspension) of the analyte in a liquid (e.g. water, possibly with a buffer added). In some embodiments, providing the analyte may comprise providing the dispersion above or below the nanopore. In some embodiments, providing the analyte may comprise providing the dispersion in the cis reservoir or in the trans reservoir.

The present disclosure is not limited by the type of analyte, provided it can be translocated through the nanopore. In some embodiments, the analyte may thus generally be any (single) molecule or (single) particle, such as in particular a charged molecule, charged particle, biomolecule or bioparticle. Notwithstanding, the analyte may in some embodiments be a polypeptide, a protein (e.g. comprising one or more polypeptides) or a nucleic acid (e.g. a deoxyribonucleic acid-DNA; or a ribonucleic acid-RNA). Such analytes may be used since the interest and usefulness for single molecule/particle detection and/or characterization is currently great for these types of bioanalytes, while e.g. compared to other techniques those of the present disclosure may be well suited therefor.

In some embodiments, translocating the analyte through the nanopore may comprise generating a potential difference between the cis and trans electrode. It will be clear that movement—and thus translocation through the nanopore—of an analyte under a potential difference can depend on its charge and the fluid convection flow which is generated. For instance, if a more positive voltage is applied to the cis electrode than to the trans electrode, a negatively charged analyte will tend to move from the trans to the cis reservoir; a positively charged analyte will tend to move from the cis to the trans reservoir; and a neutral analyte will tend to follow the fluid convection flow (induced by the movement of e.g. salt ions, and which will thus depend on these).

In some embodiments, step c may comprising sensing the electric feature at a plurality (e.g. each) of the sensing layers.

In some embodiments, sensing an electric feature in the nanopore may comprise measuring a current through the sensor. In general, the sensing layer and sensor are such that at least one electric characteristic thereof is modulated by the contents of the nanopore, which can in turn be registered as a change in signal of (e.g. a change in current through) the sensor. For example, in the case of a nanopore FET or a sensor based on a conductor layer, the resistance/conductance of the sensing layer as such may be influenced by contents of the nanopore. Conversely, in the case of an extended-gate FET, the contents of the nanopore can affect the voltage of the sensing layer—and thus the extended gate—, which will in turn change the resistance/conductance of the FET's channel region.

In some embodiments, the method may further comprise: (d) analysing the electric feature(s) sensed in step c to determine therefrom a speed and/or length of the analyte longitudinal to the nanopore. In some embodiments, step d may comprise deriving from the electrostatic potential data a plurality of event times (e.g. arrival and/or departure times) of the analyte corresponding to the plurality of sensing layers. Since the distance between sensing layers can be known, these can then be used to calculate a speed and/or a length of the analyte. This can for instance be done based on equations like:

$${v}_{a} = \frac{Δ {x}_{1 \to 2}}{Δ {f}_{1 \to 2}} and / or {l}_{a} = {v}_{a} \times Δ {t}_{a} ;$$

wherein vₐ is the speed (i.e. velocity) of the analyte longitudinal to the nanopore, Δx_{1→2} is the distance (e.g. height) between two sensing layers 1 and 2 and Δt_{1→2} is the time (i.e. duration) between registering an event of the analyte at the sensing layers 1 and 2 (e.g. the difference in arrival times, or in departure times), lₐ is the length of the analyte longitudinal to the nanopore, and Ata is the signal duration over which the analyte was detected at a sensing layer (e.g. the difference between arrival and departure times for sensing layer 1, or for sensing layer 2, or the average of these). Obviously, for more than two sensing layers, these values can be calculated multiple times (e.g., 1→2, 2→3, 1→3, etc.) and thus increases the robustness of the measurement. For example, this may be used to check whether the speed through the nanopore (or more specifically: between the sensing layers) is constant or—assuming a constant speed—to increase the accuracy of the determined value (by statistically reducing the noise through the multiple sampling).

In some embodiments, the method may further comprise: (e) determining an electric fingerprint of the analyte. In some embodiments, step e may be performed separately or together with step d. In some embodiments, determining an electric fingerprint of the analyte may comprise determining a plurality of the electric features sensed as the analyte translocates through the nanopore. The fingerprint may be used to characterize the analyte. This could for example be done by providing (e.g. experimentally or through simulations) a number of different analyte fingerprints and comparing the determined analyte fingerprint thereto, to look for any (full or partial) match.

In some embodiments, any feature of any embodiment of the third aspect may independently be as correspondingly described for any embodiment of any of the other aspects.

In a fourth aspect, the present disclosure relates to a use of a nanopore sensing device as defined in any embodiment of the first aspect, for determining a speed and/or length of an analyte longitudinal to the nanopore.

In some embodiments, any feature of any embodiment of the fourth aspect may independently be as correspondingly described for any embodiment of any of the other aspects.

The present disclosure will now be described by a detailed description of several embodiments of the present disclosure. It is clear that other embodiments of the present disclosure can be configured according to the knowledge of the person skilled in the art without departing from the true technical teaching of the present disclosure.

### Nanopore Sensing Devices Comprising a Nanopore FET

### Nanopore Sensing Devices Comprising Two Sensing Layers

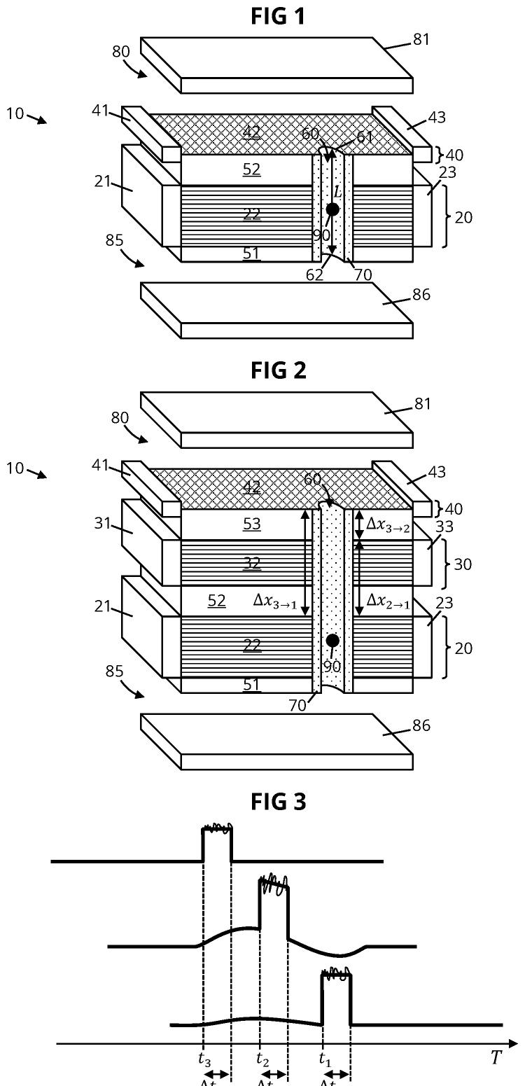

We now refer to FIG. 1, illustrating a nanopore sensing device (10) comprising two sensing layers (22, 42). Above the nanopore sensing device (10) is a cis reservoir (80) and a cis electrode (81), while below it is a trans reservoir (85) and a trans electrode (86).

The nanopore sensing device (10) itself comprises a first isolating layer (51), a first sensing layer (22), a second isolating layer (52) and a second sensing layer (42) stacked on top of one another, with a nanopore (60) crossing through the stack and opening up to the cis reservoir (80) and trans reservoir (85) on either side. An oxide (70) lines the nanopore (60) from the first isolating layer (51) to the second isolating layer (52) (i.e. not the second sensing layer (42)).

The first sensing layer (22) is a semi-conductor layer and is part of a first sensor (20), namely a nanopore FET. The nanopore FET (20) comprises a source (21) and drain (23), the first sensing layer (22) as (or at least comprising) the channel region, and is gated by (the contents of) the nanopore (60). The nanopore FET (20) may for instance be a Si nanopore FET.

The second sensing layer (42) is a conductor layer and is part of a second sensor (40). The second sensor comprises the conductive sensing layer (42) and a first (41) and second (43) contact to the latter. The resistance of the conductive sensing layer (42) is modulated by (the contents of) the nanopore (60). The conductive sensing layer (42) may for instance be graphene, a (thin) metal (e.g. Pt or Ru) or a metallic CNT.

By operating the cis (81) and trans (86) electrodes, a potential difference across the cis (80) and trans (85) reservoirs—and thus across the nanopore (60)—is generated, which can be used to translocate an analyte (90) through the nanopore (60) (cf. supra).

### Nanopore Sensing Devices Comprising Three Sensing Layers

We now refer to FIG. 2, illustrating a nanopore sensing device (10) which is generally similar to that of FIG. 1 but comprises three sensing layers (22, 32, 42).

The nanopore sensing device (10) of FIG. 2 comprises a first isolating layer (51), a first sensing layer (22), a second isolating layer (52), a second sensing layer (32), a third isolating layer (53) and a third sensing layer (42) stacked on top of one another, with a nanopore (60) crossing through the stack and opening up to the cis reservoir (80) and trans reservoir (85) on either side. An oxide (70) again lines the nanopore (60) from the first isolating layer (51) to the third isolating layer (i.e. not including third sensing layer).

The first sensing layer (22) is as described for the first sensing layer of FIG. 1.

The second sensing layer (32) is also a semi-conductor layer and is part of a second sensor (30), which is also a nanopore FET. This nanopore FET (30) comprises a further source (31) and drain (33), the second sensing layer (32) as (or at least comprising) its channel region and is also gated by (the contents of) the nanopore (60). The second nanopore FET (30) may for instance be a backend-of-the-line (BEOL) nanopore FET.

The third sensing layer (42) is as described for the second sensing layer of FIG. 1.

We now refer to FIG. 3, illustrating the signals over time (T) from the third (top), second (middle) and first (bottom) sensor one might obtain as an analyte (90) translocates through the nanopore (60) sensing device (10) as in FIG. 2 (here from the third to the first sensor). Generally each signal comprises a superposition of a background signal (here depicted as a deviation from the baseline), a main electric feature (depicted as a box pulse on top of the background signal) and a fine local variations in the dominant electric feature (depicted as a squiggle over the box). The magnitude (both in absolute terms and relative to the other features) and other characteristics of these can depend on each individual sensor. For example, the pulse height of the main feature (relative to the background) is not constant and may e.g. be lower for a conductive sensor than for a FET sensor. Likewise, some sensors may be largely sensitive only to local changes in electric field (i.e. near the sensor), while for others e.g. the background signal may be significantly determined by the contents of the nanopore as a whole.

As can be see, the three sensing layers allow to define three arrival (or departure) times t₃, t₂ and t₁; three interlayer distances Δx_{3→2}, Δx_{2→1} and Δx_{3→1} (see FIG. 2); three signal durations Δt_{a,3}, Δt_{a,2} and Δt_{a,1}; etc. And thus also to calculate three instances of the speed v_{a,3→2}, v_{a,2→1}, and v_{a,3→1}; and three instances of the length l_{a,3→2}, l_{a,2→1}, and l_{a,3→1}. By contrast, in the case of only two sensing layers, only one of each of these can be determined. Accordingly, using more than two sensing layers (e.g. three, four, etc.) allows to increase the robustness of the measurements/calculations, e.g. by statistically increasing their accuracy.

### Nanopore Sensing Devices Comprising an Extended-Gate FET

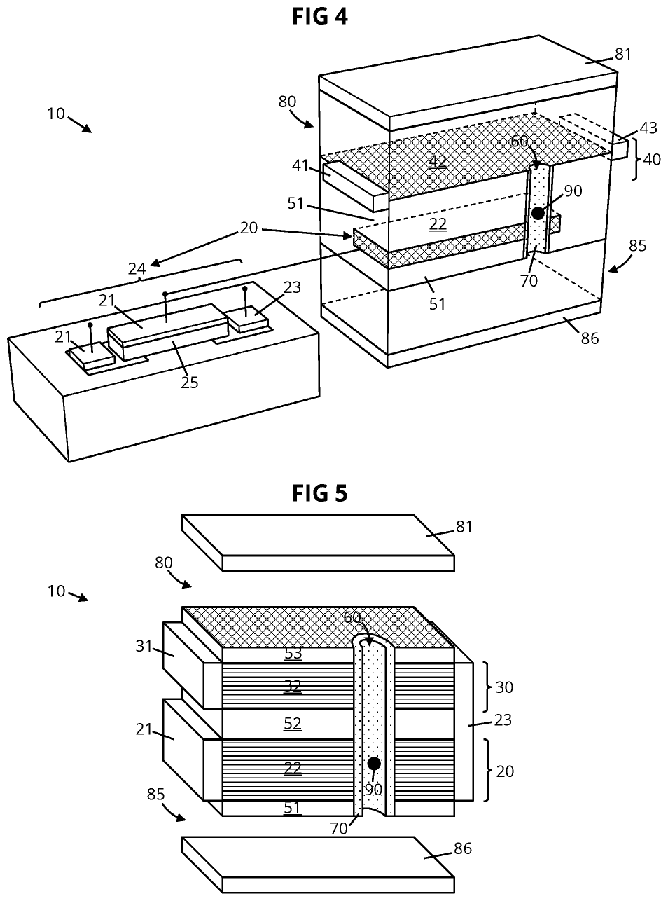

We now refer to FIG. 4, illustrating a nanopore sensing device (10) which is generally similar to that of FIG. 1 but comprises one sensing layer (22) which is part of an extended-gate FET (20).

The nanopore sensing device (10) of FIG. 4 comprises a first isolating layer (51), a first sensing layer (22), a second isolating layer (52) and a second sensing layer (42) stacked on top of one another, with a nanopore (60) crossing through the stack and opening up to the cis reservoir (80) and trans reservoir (85) on either side. An oxide (70) again lines the nanopore (60) from the first isolating layer (51) to the second isolating (i.e. not the second sensing layer (42)).

The first sensing layer (22) is a conductor layer but is part of an extended-gate FET as first sensor (20). The extended-gate FET (20) comprises a main body (24) comprising a source (21), a drain (23), a channel (25) and a gate (26). The gate (26) is however electrically coupled—and thereby extended (‘long gate’)—to the conductive first sensing layer (22). The FET's main body (24) may for instance be a Si FET, while the conductive first sensing layer (22) may for instance be graphene, a (thin) metal (e.g. Pt or Ru) or metallic CNT.

The second sensing layer (42) is as described for the second sensing layer (42) of FIG. 1.

Nanopore sensing devices wherein (some) sensor layers share a common contact
Nanopore Sensing Devices with One Common Contact

We now refer to FIG. 5, illustrating a nanopore sensing device (10) which is generally similar to that of FIG. 2 but comprises two sensing layers (22, 32) sharing a common drain (23).

The nanopore sensing device (10) of FIG. 5 comprises a first isolating layer (51), a first sensing layer (22), a second isolating layer (52), a second sensing layer (42) and a third isolating layer (53) stacked on top of one another, with a nanopore (60) crossing through the stack and opening up to the cis reservoir (80) and trans reservoir (85) on either side. An oxide (70) again lines the nanopore (60) from the first isolating layer (51) to the third isolating (53-.

The first (22) and second (32) sensing layers are generally as described for the first and second sensing layers of FIG. 2, but instead of each having their own dedicated drain they both share one common drain (23). Notwithstanding, since the first (22) and second (32) sensing layers still have individual source contacts (21, 31), they are nevertheless considered to be part of two separate sensors (20, 30).

Nanopore Sensing Devices with Two Common Contacts

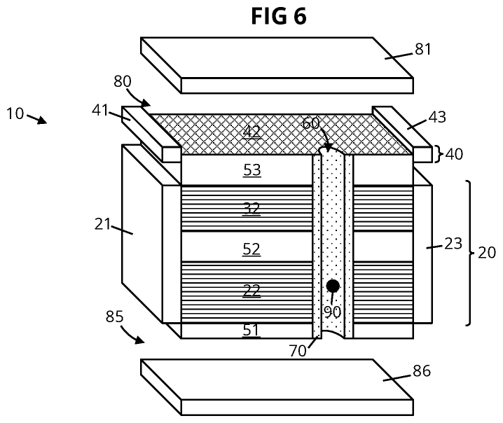

We now refer to FIG. 6, illustrating a nanopore sensing device (10) which is generally similar to that of FIG. 5 but comprises two sensing layers (22, 32) sharing both a common source (21) and common drain (23).

The nanopore (60) sensing device (10) of FIG. 6 comprises a first isolating layer (51), a first sensing layer (22), a second isolating layer (52), a second sensing layer (32), a third isolating layer (53) and a third sensing layer (42) stacked on top of one another, with a nanopore (60) crossing through the stack and opening up to the cis reservoir (80) and trans reservoir (85) on either side. An oxide (70) again lines the nanopore (60) from the first isolating layer (51) to the third isolating (53) (i.e. not the third sensing layer).

The first (22) and second (32) sensing layers generally are as described for the first and second sensing layers of FIG. 5, but now both sharing one common source (21) and one common drain (23). Since the first (22) and second (32) sensing layers now share all their contacts (21, 23), they are considered to be part of a single sensor (20).

The third sensing layer is as described for the third sensing layer of FIG. 2.

It is to be understood that although specific embodiments, specific constructions, configurations and materials have been discussed herein in order to illustrate the present disclosure. It will be apparent to those skilled in the art that various changes or modifications in form and detail may be made without departing from the scope of the present disclosure.

What is claimed is:

1. A nanopore sensing device, comprising:
a nanopore having a first orifice and second orifice, and a length running from the first to the second orifice; and
one or more sensors for sensing an electric feature in the nanopore;
wherein the nanopore sensing device comprises a plurality of sensing layers arranged along the length, each sensing layer is part of one of the sensors, and each adjacent pair of sensing layers is separated by a respective isolating layer, and wherein at least one of the sensors is a field-effect transistor.

2. The nanopore sensing device of claim 1, wherein the field-effect transistor has a subthreshold swing at room temperature that is less than or equal to 150 mV/dec.

3. The nanopore sensing device of claim 2, wherein the field-effect transistor has a subthreshold swing at room temperature that is less than or equal to 60 mV/dec.

4. The nanopore sensing device of claim 1, wherein the field-effect transistor is at least one of a nanopore field-effect transistor or an extended-gate field-effect transistor.

5. The nanopore sensing device of claim 1, wherein at least two of the sensing layers have one or two contacts in common.

6. The nanopore sensing device of claim 1, wherein at least one of the sensing layers is a conductor layer.

7. The nanopore sensing device of claim 1, wherein the isolating layers have respective thicknesses between 2 nm and 100 nm.

8. The nanopore sensing device of claim 1, wherein the nanopore has a width between 1 and 200 nm.

9. The nanopore sensing device of claim 1, wherein the nanopore has a section defined between an uppermost sensing layer and a lowermost sensing layer of the sensing layers, wherein the section has a substantially constant width and substantially uniform sidewalls.

10. The nanopore sensing device of claim 9, wherein the nanopore has a width above the uppermost sensing layer or below the lowermost sensing layer that is increased relative to the width of the nanopore between the uppermost sensing layer and the lowermost sensing layer.

11. The nanopore sensing device of claim 1, further comprising a readout circuit having a bandwidth of at least 10 kHz.

12. The nanopore sensing device of claim 1, wherein the field-effect transistor has a signal-to-noise ratio of at least 0 dB up to at least 100 kHz.

13. A system comprising:
the nanopore sensing device of claim 1; and
a microfluidic system coupled to the nanopore of the sensing device.

14. The system of claim 13, further comprising:
a cis reservoir;
a trans reservoir; and
a cis electrode and a trans electrode (86) configured to generate a potential difference to translocate an analyte through the nanopore.

15. The system of claim 13, wherein the nanopore has a section defined between an uppermost sensing layer and a lowermost sensing layer of the sensing layers of the nanopore sensing device, wherein the section has a substantially constant width and substantially uniform sidewalls.

16. The nanopore sensing device of claim 15, wherein the nanopore has a width above the uppermost sensing layer or below the lowermost sensing layer that is increased relative to the width of the nanopore between the uppermost sensing layer and the lowermost sensing layer.

17. A method for sensing an analyte, comprising:
providing an analyte in the nanopore sensing device of claim 1;
translocating the analyte through the nanopore of the nanopore sensing device; and
sensing an electric feature of the nanopore as the analyte translocates therethrough.

18. The method of claim 17, further comprising:
based on the sensed electric feature, determining at least one of a speed or a length of the analyte longitudinal to the nanopore.

19. The method of claim 17, wherein the nanopore has a section defined between an uppermost sensing layer and a lowermost sensing layer of the sensing layers of the nanopore sensing device, wherein the section has a substantially constant width and substantially uniform sidewalls.

20. The method of claim 19, wherein the nanopore has a width above the uppermost sensing layer or below the lowermost sensing layer that is increased relative to the width of the nanopore between the uppermost sensing layer and the lowermost sensing layer.
